(19) United States
(12) Patent Application Publication
(10) Pub. No.: US 2023/0321073 A1
(43) Pub. Date: Oct. 12, 2023

(54) COMPOSITIONS AND METHODS FOR TREATING METABOLIC DYSREGULATION

(71) Applicant: REDUX THERAPEUTICS, LLC, Marblehead, MA (US)

(72) Inventors: Paul Sweetnam, Marblehead, MA (US); Eric L. Keller, McLean, VA (US)

(73) Assignee: Response IP Holding Company, LLC, McLean, VA (US)

(21) Appl. No.: 18/019,715

(22) PCT Filed: Aug. 6, 2021
(86) PCT No.: PCT/US2021/044916

§ 371 Date: Feb. 3, 2023

Related U.S. Application Data
(60) Provisional application No. 63/061,899, filed on Aug. 6, 2020.

(51) Int. Cl. A61K 31/40; A61K 31/661; A61K 31/635; A61K 31/451; A61K 31/496; A61K 31/505; A61K 31/506; A61K 31/352; A61K 31/513; A61K 31/4433; A61K 31/538; A61K 31/551; A61K 31/5415; A61K 31/495; A61K 31/445; A61K 31/47; A61K 31/426; A61K 31/135; A61K 31/665; A61K 31/4725; A61K 31/683; A61K 31/454; A61K 31/382; A61K 31/438; A61K 31/5377; A61K 31/542; A61K 31/573; A61K 31/4418; A61K 31/52; A61K 31/553; A61K 45/06

(52) U.S. Cl. CPC A61K 31/47; A61K 31/135; A61K 31/352; A61K 31/382; A61K 31/40; A61K 31/426; A61K 31/438; A61K 31/4418; A61K 31/4433; A61K 31/445; A61K 31/451; A61K 31/454; A61K 31/4725; A61K 31/495; A61K 31/496; A61K 31/505; A61K 31/506; A61K 31/513; A61K 31/52; A61K 31/5377; A61K 31/538; A61K 31/5415; A61K 31/542; A61K 31/551; A61K 31/553; A61K 31/573; A61K 31/635; A61K 31/661; A61K 31/665; A61K 31/683; A61K 45/06

(57) ABSTRACT

In certain embodiments, the present disclosure relates to compositions comprising a compound that modulates the activity of microsomal triglyceride transfer protein (MTP), and therapeutic methods of using such compositions.

### BACKGROUND

Metabolic dysregulation in patients taking second-generation antipsychotic medications, and antiretroviral therapies such a HIV protease inhibitors, is a documented problem. See, e.g., Fenton, W.S., Am. J. Psychiatry 2006, 163(10); and Geletko, S.M., Am. J. Health Syst. Pharm. 2001, 58(7). Microsomal triglyceride transfer protein (MTP) is a member of a group of proteins that bind and shuttle individual lipids between membranes, and is present in high concentrations in the lumen of the endoplasmic reticulum of enterocytes. MTP is an attractive pharmacological target, with inhibition having the potential to achieve both lipid lowering and anti-inflammatory effects. However, despite clinical evidence of efficacy, all first generation MTP inhibitors have exhibited serious toxicology problems related to liver exposure, including elevated liver enzymes and hepatic steatosis. There remains a need for safe and effective methods for treating metabolic dysregulation.

### SUMMARY

In one aspect, provided herein is a method of treating a disorder in a subject resulting from activation of one or more xenobiotic sensor receptors in the subject by a xenobiotic agent, comprising administering to the subject an effective amount of a composition comprising a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion.

In another aspect, provided herein is a pharmaceutical composition for treating a disorder in a subject resulting from activation of one or more xenobiotic sensor receptors in the subject by a xenobiotic agent, the pharmaceutical composition comprising an effective amount of a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion.

In another aspect, provided herein is a pharmaceutical composition comprising a first compound that is an omega-3 fatty acid, or a prodrug thereof, and second compound that is an inhibitor of microsomal triglyceride transfer protein (MTP).

In yet another aspect, provided herein is a method of treating a disease or disorder in a subject, comprising administering to the subject a first compound that is an omega-3 fatty acid, or a prodrug thereof, and second compound that is an inhibitor of microsomal triglyceride transfer protein (MTP).

### BRIEF DESCRIPTION OF THE DRAWINGS

FIG. 1 depicts the treatment of xenobiotic-induced hyperlipidemia using a gut-selective MTP inhibitor.

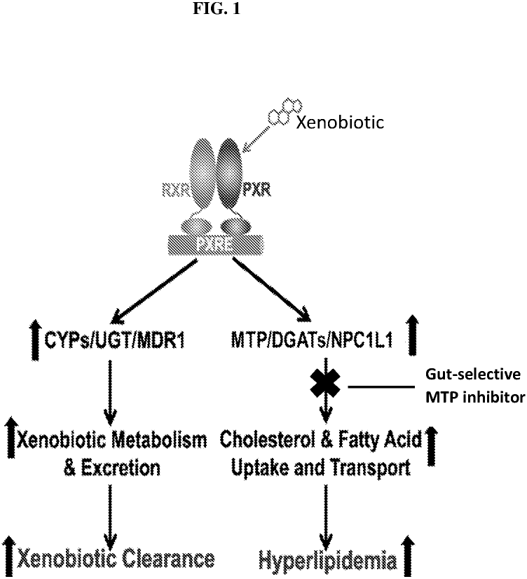

FIG. 2 lists classes of drugs that are known to be associated with dyslipidemia.

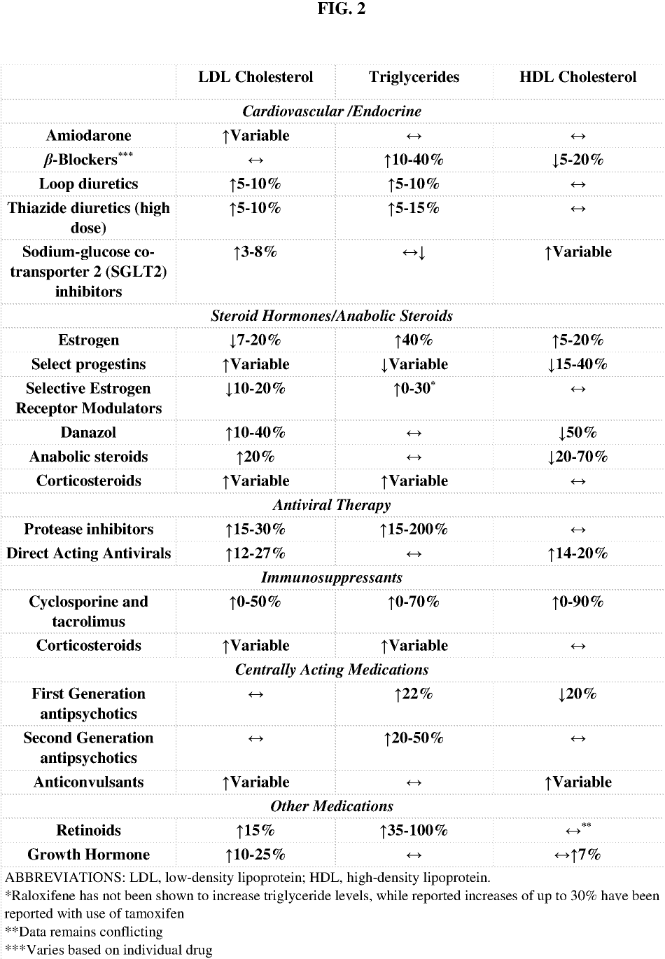

### DETAILED DESCRIPTION

In one aspect, provided herein is a method of treating a disorder in a subject resulting from activation of one or more xenobiotic sensor receptors in the subject by a xenobiotic agent, comprising administering to the subject an effective amount of a composition comprising a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion.

In certain embodiments, the disorder is related to loss of intestinal homeostasis, an inflammatory disorder, or an autoimmune disease. In certain embodiments, the disorder related to loss of intestinal homeostasis is dyslipidemia, hyperlipidemia, metabolic syndrome, or a lipid-related metabolic disorder. In certain embodiments, the inflammatory disorder is post-prandial related inflammation.

In certain embodiments, administration of the composition to the subject results in inhibition of absorption, assembly, and/or transport of lipids, cholesterol, and/or microbial metabolites in the GI tract of the subject. In certain embodiments, the lipids are selected from diglycerides, triglycerides, fatty acids, phospholipids, cholesterol, cholesterol esters, glycolipids, bile acids, and microbial metabolitcs. In certain embodiments, the microbial metabolites are selected from glycosaccharides, glycolipids, free fatty acids, and microbial peptides.

In certain embodiments, the method further comprising administering the xenobiotic agent to the subject. In certain embodiments, the composition and the xenobiotic agent are administered to the subject at the same time. In certain embodiments, the composition is administered to the subject after administration of the xenobiotic agent. In certain embodiments, the composition is administered to the subject before administration of the xenobiotic agent.

### Xenobiotic Agents and Receptors

A “xenobiotic” or “xenobiotic agent” is a chemical substance found within an organism that is not naturally produced or expected to be present within the organism. It can also cover substances that are present in much higher concentrations than are usual.

Natural compounds can also become xenobiotics if they are taken up by another organism, such as the uptake of natural human hormones by fish found downstream of sewage treatment plant outfalls, or the chemical defenses produced by some organisms as protection against predators. In certain embodiments, the xenobotic agent has an EC50 of less than 10 µM in a cellular PXR assay. See, e.g., Shukla, S.J. et al., Drug Metabolism and Disposition 2011, 39, 151. In certain embodiments, the xenobiotic agent causes a gut-specific increase of the level of MTP. In certain embodiments, the xenobiotic agent activates or induces one or more cytochrome P450 enzymes. The body removes xenobiotics by xenobiotic metabolism. This consists of the deactivation and the excretion of xenobiotics, and happens mostly in the liver. Excretion routes are urine, feces, breath, and sweat. Hepatic enzymes are responsible for the metabolism of xenobiotics by first activating them (oxidation, reduction, hydrolysis and/or hydration of the xenobiotic), and then conjugating the active secondary metabolite with glucuronic acid, sulfuric acid, or glutathione, followed by excretion in bile or urine. An example of a group of enzymes involved in xenobiotic metabolism is hepatic microsomal cytochrome P450. See, e.g., Tebbens, J.D. et al., Int. J. Mol. Sci. 2018, 19, 1785. These enzymes that metabolize xenobiotics are very important for the pharmaceutical industry, because they are responsible for the breakdown of medications. Although the body is able to remove xenobiotics by reducing it to a less toxic form through xenobiotic metabolism then excreting it, it is also possible for it to be converted into a more toxic form in some cases. This process is referred to as bioactivation and can result in structural and functional changes to the microbiota. Exposure to xenobiotics can disrupt the microbiome community structure, either by increasing or decreasing the size of certain bacterial populations depending on the substance. Functional changes that result vary depending on the substance and can include increased expression in genes involved in stress response and antibiotic resistance, and changes in the levels of metabolites produced.

In certain embodiments, the xenobiotic agent is a compound that is known to cause dyslipidemia, selected from amiodarone, β-blockers, loop diuretics, and thiazide diuretics.

In certain embodiments, the xenobiotic agent is selected from phenothiazines, thioxanthenes, benztropines, corticosteroids, azoles, dihydropyridines, thiazolidinediones, thiazides, leptin, and leptin mimetics. In certain particular embodiments, the xenobiotic agent is selected from rifampicin, dexamethasone, ambrisentan, amlodipine, atorvastatin, bosentan, bumecainum, ciglitazone, clofenvinfosum, colforsin, demecolcine, dibunate, diclazuril, docusate, dronabinol, eburnamonine, ecopipamum, famprofazone, felodipine, flurometholone, fluvastatin, loratadine, lovastatin, metolazone, nilvadipine, nisoldipine, oxatomide, plicamycin, propiconazole, rifaximin, rimexolone, riodipine, simvastatin, spiroxatrine, teniliodona, terconanzole, testosterone, troglitazone, and zafirlukast.

In certain embodiments, the xenobiotic agent is an intestinal activator of STAT3 and/or MAPK.

In certain embodiments, the xenobiotic agent is a non-nucleoside reverse transcriptase inhibitor, an antiretroviral agent, or a combination thereof. In certain particular embodiments, the xenobiotic agent is selected from lopinavir, atazanavir, fosamprenavir, saquinavir, darunavir, tipranavir, efavirenz, nevirapine, tenofovir, abacavir, zidovudine, stavudine, ritonavir, amprenavir, indinavir, and nelfinavir.

In certain embodiments, the xenobiotic agent is an antipsychotic agent. In certain particular embodiments, the antipsychotic agent is selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine, zuclopenthixol, amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone, and zotepine.

A “xenobiotic-sensing receptor” or “xenobiotic receptor” is a receptor that binds a xenobiotic agent. See, e.g., Mackowiak, et al., Drug Metabolism and Disposition September 2018, 46 (9) 1361-1371. In certain embodiments, the one or more xenobiotic sensor receptors are selected from pregnane X receptor (PXR) and constitutive active/androstane receptor (CAR). See, e.g., Timsit et al., Steroids. 2007, 72 (3): 231-46.

### Compounds

The methods provided herein comprise administering to the subject an effective amount of a composition comprising a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion. Such compounds are described, for example, in U.S. Pat. Nos. 8,980,915 and 9,656,960, the contents of which are herein incorporated by reference in their entirety.

In certain embodiments, the compound has the structure of Formula (I):

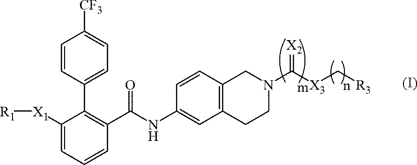

or a pharmaceutically acceptable salt, solvate, ester or hydrate thereof, wherein:
R₁ is alkyl, cycloalkyl, heterocyclyl, or R₄R₅NC(O)CH₂;
X₁ is a direct bond, O, S, N(R₆), C(O)NR₆, or N(R₆)C(O);
X₂ is O, N(R₆), or S;
X₃ is a direct bond, O, N(R₆) CH₂, arylene, or S;
R₃ is H, alkyl, alkoxy, heteroalkyl, cycloalkyl, heterocyclyl, aryl, heteroaryl, aryloxy, alkoxycarbonyl, arylcarbonyl, aryloxycarbonyl, —OH, —SH, or NR₄Rs;
R₄ and R₅ are, independently for each occurrence, H, alkyl, cycloalkyl, heterocyclyl, aryl, heteroaryl, heteroalkyl, aralkyl, aminocarbonyl, alkylcarbonyl, alkoxycarbonyl, arylcarbonyl, or aryloxycarbonyl;
R₆ is, independently for each occurrence, H or alkyl;
m is 0 or 1; and
n is an integer from 0 to 3;
provided that if m is 0, X₃ is a direct bond or CH₂.

In certain embodiments, X₁ is O.

In certain embodiments, R₁ is alkyl. In certain embodiments, R₁ is methyl. In certain embodiments, R₁ and X₁ taken together form a moiety selected from (C₁-C₆-alkyl)—O—;

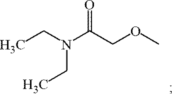

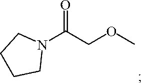

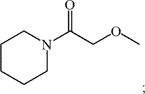

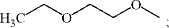

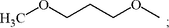

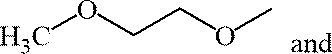

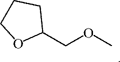

In certain embodiments, R₁ and X₁ taken together form H₃C—O—, CH₃CH₂—O—, or (CH₃)₂CH—O—.

In certain embodiments, R₃ is aryl. In certain embodiments, R₃ is substituted or unsubstituted phenyl.

In certain embodiments, m is 1.

In certain embodiments, the moiety:

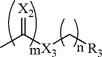

represents one of the following groups:

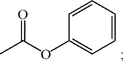

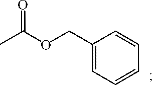

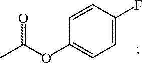

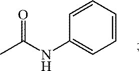

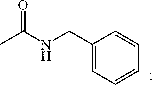

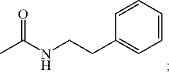

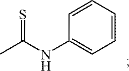

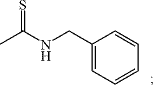

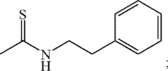

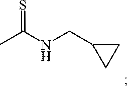

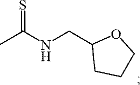

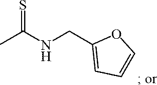

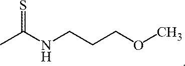

In certain embodiments, the compound has the structure of Formula (II):

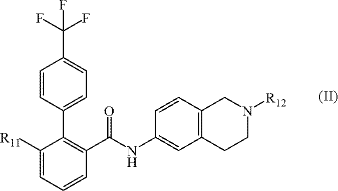

or a pharmaceutically acceptable salt, ester, isomer, or hydrate thereof, wherein:
R₁₁ is H or alkyl—O—, wherein the alkyl is substituted or unsubstituted; and
R₁₂ is substituted or unsubstituted heteroalkyl.

In certain embodiments, R₁₁ is:

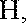

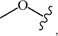

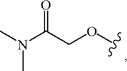

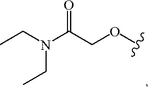

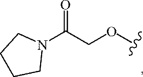

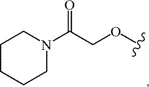

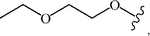

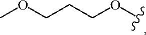

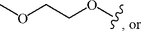

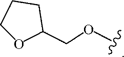

In certain embodiments, R₁₂ is:

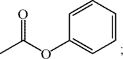

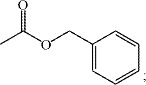

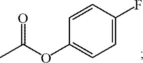

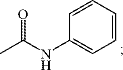

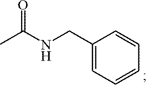

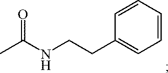

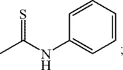

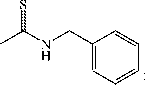

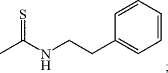

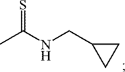

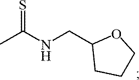

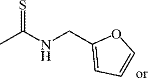

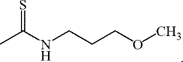

In certain embodiments, the compound has a structure selected from:

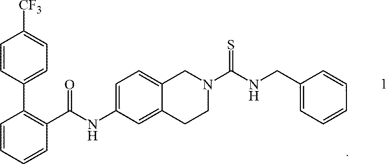

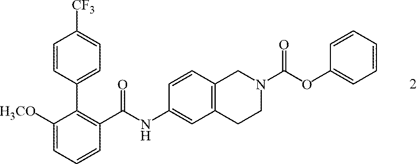 [SMILES: COc1cccc(C(=O)Nc2ccc3c(c2)CCN(C(=O)Oc2ccccc2)C3)c1-c1ccc(C(F)(F)F)cc1]

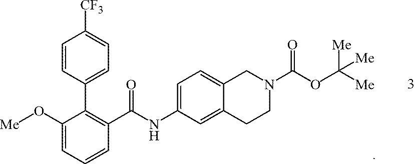

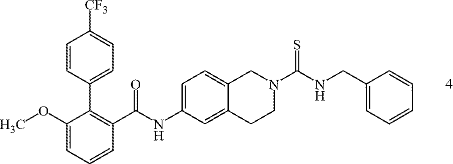 [SMILES: COc1cccc(C(=O)Nc2ccc3c(c2)CCN(C(=S)NCc2ccccc2)C3)c1-c1ccc(C(F)(F)F)cc1]

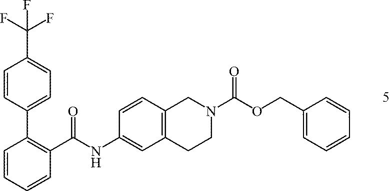 [SMILES: O=C(Nc1ccc2c(c1)CCN(C(=O)OCc1ccccc1)C2)c1ccccc1-c1ccc(C(F)(F)F)cc1]

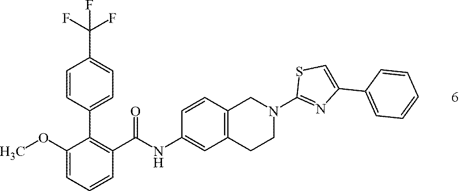 [SMILES: COc1cccc(C(=O)Nc2ccc3c(c2)CCN(c2nc(-c4ccccc4)cs2)C3)c1-c1ccc(C(F)(F)F)cc1]

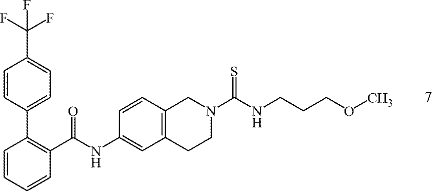 [SMILES: COCCCNC(=S)N1CCc2cc(NC(=O)c3ccccc3-c3ccc(C(F)(F)F)cc3)ccc2C1]

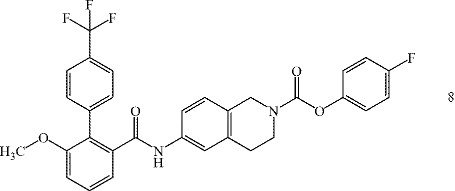 [SMILES: COc1cccc(C(=O)Nc2ccc3c(c2)CCN(C(=O)Oc2ccc(F)cc2)C3)c1-c1ccc(C(F)(F)F)cc1]

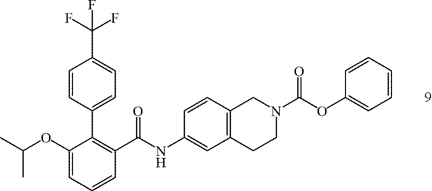 [SMILES: CC(C)Oc1cccc(C(=O)Nc2ccc3c(c2)CCN(C(=O)Oc2ccccc2)C3)c1-c1ccc(C(F)(F)F)cc1]

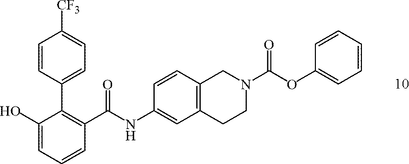 [SMILES: O=C(Nc1ccc2c(c1)CCN(C(=O)Oc1ccccc1)C2)c1cccc(O)c1-c1ccc(C(F)(F)F)cc1]

and pharmaceutically acceptable salts, solvates, hydrates, or esters thereof.

In certain embodiments, the compound is diethyl 2-((3-dimethylcarbamoyl-4-((4′-trifluoromethylbiphenyl-2-carbonyl)amino)phenyl)acetyloxymethyl)-2-phenylmalonate, or a pharmaceutically acceptable salt, solvate, hydrate, or ester thereof.

In certain embodiments, the compound is a GI selective MTP inhibitor.

In certain embodiments, the compound does not inhibit the activity of PXR. In certain embodiments, the compound does not substantially inhibit the activity of PXR.

In certain embodiments, the compound does not activate or induce a cytochrome P450 enzyme.

In certain embodiments, the compound is a systemically available inhibitor of MTP, NPC1N1, DGAT, MGAT, or ApoB secretion, and wherein the composition is formulated to limit oral bioavailability of the compound.

In certain embodiments, the compound is a systemically available inhibitor of MTP, NPC1N1, DGAT, MGAT, or ApoB secretion, and wherein the composition is formulated to promote GI selectivity of the compound.

In certain embodiments, the compound has less than 10% oral bioavailability. In certain embodiments, the compound has less than 3% oral bioavailability. In certain embodiments, the compound has less than 1% oral bioavailability.

### Compositions

In certain embodiments, the composition is formulated for immediate release. In certain embodiments, in the composition is formulated for extended release.

In certain embodiments, the composition further comprises an additional therapeutic agent. In certain embodiments, the additional therapeutic agent modulates the absorption, assembly, and/or transport of lipids, cholesterol, and/or microbial metabolites in the GI tract of the subject. In certain embodiments, the additional therapeutic agent is ezetimibe. In certain embodiments, the additional therapeutic agent is a bile acid sequestrant. In certain embodiments, the bile acid sequestrant is selected from colestipol and cholestryramine.

In certain embodiments, the additional therapeutic agent modulates production, secretion, transport, and/or homeostasis of lipids in the body of the subject. In certain embodiments, the additional therapeutic agent is an HMG-CoA reductase inhibitor, a fibric acid analog, nicotinic acid, an omega-3 fatty acid, or a PCSK9 inhibitor.

In certain embodiments, the additional therapeutic agent modulates the activation of the 5′ adenosine monophosphate-activated protein kinase (AMPK) pathway in the GI tract of the subject. In certain embodiments, the additional therapeutic agent is metformin, or a pharmaceutically acceptable salt or derivative thereof.

In certain embodiments, the additional therapeutic agent is bempedoic acid, or a pharmaceutically acceptable prodrug or salt thereof.

In certain embodiments, the additional therapeutic agent is a polyphenol. In certain embodiments, the polyphenol is epigallocatechin gallate, quercetin, apigenin, resveratrol, berberine, carnosol, curcumin, thienopyridone, salicylic acid, or a pharmaceutically acceptable salt, ester or derivative thereof. In certain embodiments, the polyphenol is troglitazone, S17834 ([6,8-diallyl 5,7-dihydroxy 2-(2-allyl 3-hydroxy 4-methoxyphenyl)1-H benzo(b)pyran-4-one]), or MT 63-78 (CAS No.: 1179347-65-9). See, for example, International Patent Publication Number WO 2003/05993, the contents of which is herein incorporated by reference in its entirety.

In certain embodiments, the additional therapeutic agent is a biguanide, a thiazolidinedione, or a Rho kinase inhibitor. See, for example, International Patent Publication Numbers WO 2008/054599 and WO 2015/054317, the contents of which are herein incorporated by reference in their entirety.

In certain embodiments, the additional therapeutic agent is an AMPK activator selected from: PF-249 (CAS No.: 1467059-70-6), PF-739 (CAS No.: 1852452-14-2), MK-8722 (CAS No.: 1394371-71-1), AICAR (N¹-(β-D-Ribofuranosyl)-5-aminoimidazole-4-carboxamide), A-769662 (6,7-Dihydro-4-hydroxy-3-(2′-hydroxy[1,1′-biphenyl]-4-yl)-6-oxo-thieno[2,3-b]pyridine-5-carbonitrile), cryptotanshinone (1,2,6,7,8,9-Hexahydro-1,6,6-trimethyl[1,2-b]furan-10,11-dione), RSVA 405 (2-[[4-(Diethylamino)-2-hydroxyphenyl]methylene]hydrazide-4-pyridinecarboxylic acid), ZLN 024 (2-[[2-(2-Bromo-4-methylphenoxy)ethyl]thiolpyrimidine), PT-1 (2-Chloro-5-[[5-[[5-(4,5-Dimethyl-2-nitrophenyl)-2-furanyl]methylene]-4,5-dihydro-4-oxo-2-thiazolyl]amino]benzoic acid), PF-06409577 (6-Chloro-5-[4-(1-hydroxycyclobutyl)phenyl]-1H-indole-3-carboxylic acid), α-lipoic acid, C-2 (benzimidazole, 5-(5-hydroxyl-isoxazol-3-yl)-furan-2-phosphonic acid), C-13 (prodrug of C-2; See, for example, Hu et al., Tumour Biol. 2016 Jan;37(1):1071-8), Compound 991 (CAS No.: 1219739-36-2), and ginsenosides.

Other AMPK activators are disclosed in International Patent Publication Numbers WO 2009/124636, WO 2009/100130, WO 2011/029855, WO 2011/138307, WO 2011/080277, WO 2011/032320, and WO 2011/033099, the contents of which are herein incorporated by reference in their entirety.

In certain embodiments, the additional therapeutic agent is a CD1d modulator, i.e., a compound that modulates CD1d function. In certain embodiments, the additional therapeutic agent is a CD1d inhibitor, i.e., a compound that inhibits CD1d function. In certain embodiments, CD1d modulator or inhibitor is an anti-inflammatory drug.

In certain embodiments, the anti-inflammatory drug is a non-steroidal anti-inflammatory drug (NSAID), a corticosteroid (e.g., budesonide), an aminosalicylate (e.g., 5-ASA, and mesalamine), or an antibody (e.g., mepolizumab, adalimumab, golimumab, certolizumab, infliximab, tysbari, vedolizumab, and ustekinumab).

In certain embodiments, the additional therapeutic agent is an immune system suppressing drug. In certain embodiments, the immune system suppressing drug is azathioprine, mercaptopurine, cyclosporine, or methotrexate.

In certain embodiments, the additional therapeutic agent is a TNF-α inhibitor, a dipeptydilpeptidase- 4 (DPP-4) inhibitor (e.g., sitagliptin), or a sodium-glucose co-transporter 2 (SGLT2) inhibitor.

### Pharmaceutical Compositions, Administration and Dosage

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

In certain embodiments, the pharmaceutical composition is formulated for oral administration. Solid dosage forms for oral administration include capsules, tablets, pills, powders, and granules. In certain embodiments, the pharmaceutical composition is formulated for enteric delivery. Compounds provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

In certain embodiments, the pharmaceutical composition is formulated for controlled release within the lower intestine or colon of a subject. Such a pharmaceutical composition may be further formulated for enteric delivery. Solid dosage forms of the compositions (e.g., pharmaceutical compositions) of the invention may optionally be scored or prepared with coatings and shells, such as enteric coatings and other coatings. They may also be formulated so as to provide slow or sustained release of the active ingredients therein using, for example, ethyl cellulose, hydroxypropyl cellulose, hydroxypropyl methyl cellulose, and combinations thereof, in varying proportions to provide the desired release profile, other polymer matrices. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredients only in a certain portion of the gastrointestinal tract, optionally, in a delayed manner. Examples of embedding compositions which can be used include polymeric substances.

A coating on a solid dosage form (e.g., to achieve controlled or sustained release) may be applied in the form of an organic or aqueous solution or dispersion. The coating may be applied to obtain a weight gain from about 1 to about 25% of the substrate in order to obtain a desired sustained release profile or controlled-release profile. Such formulations are described, e.g., in detail in U.S. Pat. Nos. 5,273,760 and 5,286,493; both incorporated herein by reference in their entirety. Other examples of controlled and sustained release formulations and coatings which may be used in accordance with the invention include U.S. Pat. Nos. 5,324,351; 5,356,467, and 5,472,712; all of which are herein incorporated by reference in their entirety.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

In certain embodiments, the effective amount of the composition contains a dose of the compound in the range of about 0.1 to about 5000 mg. For example, the dose of the compound may be in the range of 0.1-10 mg, 0.1-50 mg, 0.1-100 mg, 0.1-200 mg, 0.1-400 mg, 0.1-800 mg, 0.1-1200 mg, 0.1-2000 mg, 0.1-3000 mg, or 0.1-4000 mg. The dose of the compound may be in the range of 1-10 mg, 1-50 mg, 1-100 mg, 1-200 mg, 1-400 mg, 1-800 mg, 1-1200 mg, 1-2000 mg, 1-3000 mg, or 1-4000 mg. The dose of the compound may be in the range of 10-50 mg, 10-100 mg, 10-200 mg, 10-400 mg, 10-800 mg, 10-1200 mg, 10-2000 mg, 10-3000 mg, or 10-4000 mg. The dose of the compound may be in the range of 100-200 mg, 100-400 mg, 100-800 mg, 100-1200 mg, 100-2000 mg, 100-3000 mg, or 100-4000 mg. The dose of the compound may be in the range of 200-1000 mg, 400-1000 mg, 800-1000 mg, 1000-2000 mg, 2000-3000 mg, or 3000-4000 mg.

In certain embodiments, the effective amount of the composition contains a dose of the compound in the range of about 1 to about 1200 mg/kg body weight. In certain embodiments, the effective amount of the composition contains a dose of the compound in the range of about 5 to about 800 mg/kg body weight. In certain embodiments, the effective amount of the composition contains a dose of the compound in the range of about 0.1 to about 15 mg/kg body weight. In certain embodiments, the effective amount of the composition contains a dose of the compound in the range of about 1 to about 5 mg/kg body weight.

In another aspect, provided herein is a pharmaceutical composition comprising compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion, wherein the compound is as defined in any embodiment above. In a particular embodiment, the compound is compound 2:

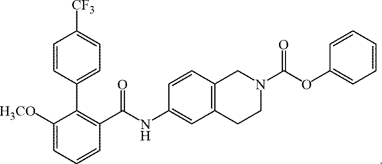

In another aspect, provided herein is a kit comprising a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion, wherein the compound is as defined in any embodiment above; and optionally an additional therapeutic agent as defined in any embodiment above.

### Polyunsaturated Fatty Acid Compositions

In another aspect, provided herein is a pharmaceutical composition comprising a first compound that is a polyunsaturated fatty acid (for example, an omega-3 fatty acid, or a prodrug thereof), and second compound that is an inhibitor of microsomal triglyceride transfer protein (MTP).

In certain embodiments, the first compound is linoleic acid (ALA), eicosapentaenoic acid (EPA), docosapentaenoic acid (DPA), docosahexaenoic acid (DHA), or a prodrug thereof. In certain embodiments, the first compound is two or more omega-3 fatty acids, or prodrugs thereof.

In certain embodiments, eicosapentaenoic acid, or a prodrug thereof, is present in an amount of about 70% to about 90%, by weight, of all fatty acids or prodrugs thereof present in the pharmaceutical composition. In certain embodiments, docosapentaenoic acid, or a prodrug thereof, is present in an amount up to about 10%, by weight, of all fatty acids present in the pharmaceutical composition. In certain embodiments, docosapentaenoic acid, or a prodrug thereof, is present in an amount up to about 5%, by weight, of all fatty acids present in the pharmaceutical composition. In certain embodiments, docosapentaenoic acid, or a prodrug thereof, is present in an amount of about 5%, by weight, of all fatty acids present in the pharmaceutical composition.

In certain embodiments, the prodrug form of the polyunsaturated fatty acid (for example, omega-3 fatty acid) is an ester. In certain embodiments, the ester is a substituted or unsubstituted alkyl ester, or a substituted or unsubstituted heteroalkyl ester. In certain embodiments, the ester is an unsubstituted alkyl ester. In certain embodiments, the unsubstituted alkyl ester is a methyl ester, ethyl ester, propyl ester, isopropyl ester, n-butyl ester, or isobutyl ester.

In certain embodiments, the second compound is a small molecule, a polypeptide, or a polynucleotide. In a particular embodiment, the second compound is a small molecule.

In certain embodiments, the second compound has the structure of Formula (I):

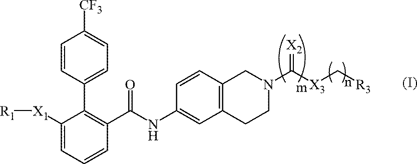

or a pharmaceutically acceptable salt, solvate, ester or hydrate thereof, wherein:
R₁ is alkyl, cycloalkyl, heterocyclyl, or R₄R₅NC(O)CH₂;
X₁ is a direct bond, O, S, N(R₆), C(O)NR₆, or N(R₆)C(O);
X₂ is O, N(R₆), or S;
X₃ is a direct bond, O, N(R₆) CH₂, arylene, or S;
R₃ is H, alkyl, alkoxy, heteroalkyl, cycloalkyl, heterocyclyl, aryl, heteroaryl, aryloxy, alkoxycarbonyl, arylcarbonyl, aryloxycarbonyl, —OH, —SH, or NR₄Rs;
R₄ and R₅ are, independently for each occurrence, H, alkyl, cycloalkyl, heterocyclyl, aryl, heteroaryl, heteroalkyl, aralkyl, aminocarbonyl, alkylcarbonyl, alkoxycarbonyl, arylcarbonyl, or aryloxycarbonyl;
R₆ is, independently for each occurrence, H or alkyl;
m is 0 or 1; and
n is an integer from 0 to 3;
provided that if m is 0, X₃ is a direct bond or CH₂.

In certain embodiments, the second compound has the structure of Formula (II):

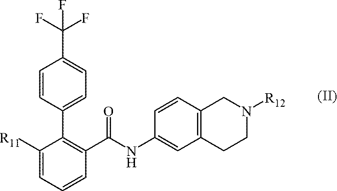

or a pharmaceutically acceptable salt, ester, isomer, or hydrate thereof, wherein:
R₁₁ is H or alkyl—O—, wherein the alkyl is substituted or unsubstituted; and
R₁₂ is substituted or unsubstituted heteroalkyl.

In certain embodiments, the second compound has a structure selected from compounds 1, 2, 3, 4, 5, 6, 7, 8, 9, 10, and pharmaceutically acceptable salts, solvates, hydrates, or esters thereof.

In certain embodiments, the second compound is diethyl 2-((3-dimethylcarbamoyl-4-((4′-trifluoromethylbiphenyl-2-carbonyl)amino)phenyl)acetyloxymethyl)-2-phenylmalonate, or a pharmaceutically acceptable salt, solvate, hydrate, or ester thereof.

In certain embodiments, the second compound is GI selective. In certain embodiments, the second compound does not inhibit the activity of PXR. In certain embodiments, the compound does not substantially inhibit the activity of PXR.

In certain embodiments, the composition is formulated for immediate release. In certain embodiments, the composition is formulated for extended release, as described herein.

In certain embodiments, the weight percent of the first compound in the composition is 1-5%, 5-7%, 7-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-93%, 93-95%, or 95-99%. In certain embodiments, the weight percent of the second compound in the composition is 1-5%, 5-7%, 7-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-93%, 93-95%, or 95-99%.

In certain embodiments, the composition comprises the first compound in an amount of about 10 mg to about 5000 mg. For example, the amount of the first compound may be in the range of 10-50 mg, 10-100 mg, 10-200 mg, 10-400 mg, 10-800 mg, 10-1200 mg, 10-2000 mg, 10-3000 mg, or 10-4000 mg. The amount of the first compound may be in the range of 100-200 mg, 100-400 mg, 100-800 mg, 100-1200 mg, 100-2000 mg, 100-3000 mg, 100-4000 mg, or 100-5000 mg. The amount of the first compound may be in the range of 200-1000 mg, 400-1000 mg, 800-1000 mg, 1000-2000 mg, 2000-3000 mg, 3000-4000 mg, or 4000-5000 mg.

In a particular embodiment, the composition comprises eicosapentaenoic acid in an amount of about 750 mg to about 950 mg. In another particular embodiment, the composition comprises the second compound in an amount of about 1 mg to about 1200 mg.

In another aspect, provided herein is a method of treating a disease or disorder in a subject in need thereof, comprising administering to the subject a first compound that is an omega-3 fatty acid, or a prodrug thereof, and second compound that is an inhibitor of microsomal triglyceride transfer protein (MTP).

In certain embodiments, the disease or disorder is a metabolic disease. For example, in certain embodiments, the metabolic disease is hypertriglyceridemia, mixed dyslipidemia, atherosclerosis, obesity, or diabetes.

In certain embodiments, the first compound is linoleic acid (ALA), eicosapentaenoic acid (EPA), docosahexaenoic acid (DHA), or a prodrug thereof. In certain embodiments, the first compound is two or more omega-3 fatty acids, or prodrugs thereof.

In certain embodiments, the prodrug is an ester form of the first compound. In certain embodiments, the ester is a substituted or unsubstituted alkyl ester, or a substituted or unsubstituted heteroalkyl ester. In certain embodiments, the ester is an unsubstituted alkyl ester. In certain embodiments, the unsubstituted alkyl ester is a methyl ester, ethyl ester, propyl ester, isopropyl ester, n-butyl ester, or isobutyl ester.

In certain embodiments, the second compound is a small molecule, a polypeptide, or a polynucleotide. In certain embodiments, the second compound is a small molecule.

In certain embodiments, the second compound has the structure of Formula (I), as defined herein, or a pharmaceutically acceptable salt, solvate, ester or hydrate thereof.

In certain embodiments, the second compound has the structure of Formula (II), as defined herein, or a pharmaceutically acceptable salt, solvate, ester or hydrate thereof.

In certain embodiments, the second compound has a structure selected from compounds 1, 2, 3, 4, 5, 6, 7, 8, 9, 10, and pharmaceutically acceptable salts, solvates, hydrates, or esters thereof.

In certain embodiments, the second compound is diethyl 2-((3-dimethylcarbamoyl-4-((4′-trifluoromethylbiphenyl-2-carbonyl)amino)phenyl)acetyloxymethyl)-2-phenylmalonate, or a pharmaceutically acceptable salt, solvate, hydrate, or ester thereof.

In certain embodiments, the second compound is GI selective.

In certain embodiments of the method, the first compound is administered prior to the second compound, e.g., less than hour prior, between 1-2 hours prior, between 2-4 hours prior, between 4-8 hours prior, between 8-16 hours prior, or between 16-48 hours prior. In other embodiments, the first compound is administered after the second compound, e.g., less than hour after, between 1-2 hours after, between 2-4 hours after, between 4-8 hours after, between 8-16 hours after, or between 16-48 hours after.

In certain embodiments, the first compound and the second compound are administered simultaneously. In certain particular embodiments, the method comprises administering to the subject a pharmaceutical composition comprising a first compound that is an polyunsaturated fatty acid (for example, an omega-3 fatty acid, or a prodrug thereof), and second compound that is an inhibitor of microsomal triglyceride transfer protein (MTP), as described in various embodiments herein.

In certain embodiments, the subject has a history of acute heart failure, atrial fibrillation, hypoalbuminemia, or high inflammatory activity.

In certain embodiments, said treating comprises reducing serum triglycerides in the subject. In certain embodiments, serum triglycerides are reduced as compared to a fasted state. In certain embodiments, serum triglycerides are reduced as compared to a non-fasted state. In certain embodiments, serum triglycerides are reduced as compared to an average serum triglyceride level over a period of time, such as 24 hours, 48 hours, 72 hours, 96 hours, a week, two weeks, a month, two months, six months, or a year.

In certain embodiments, the treatment comprises two or more administrations of the first compound and the second compound per day.

In certain embodiments, the treatment comprises one or more administrations of the first compound per day and two more administrations of the second compound per day. In certain embodiments, the treatment comprises two or more administrations of the first compound per day and one more administrations of the second compound per day.

In certain embodiments, the efficacy of the first compound is improved by administration of the second compound, as compared to the efficacy of the first compound alone. For example, said efficacy of the first compound is the ability of the first compound to lower serum triglycerides in the subject.

### Definitions

The terms “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

As used herein, the terms “activate” and “activation” in the context of a biological receptor, refer to the promotion of the function of the receptor via the direct, indirect, or allosteric interaction of an agent on the receptor.

As used herein, the term “modulate” in the context of a biological receptor, refers to the act of quantitatively changing, for example, increasing or decreasing, or qualitatively changing the function of the receptor via the direct, indirect, or allosteric interaction of an agent on the receptor.

The term “inflammation” refers to any types of inflammation, such those caused by the immune system (immune-mediated inflammation) and by the nervous system (neurogenic inflammation), and any symptom of inflammation, including redness, heat, swelling, pain, loss of function, and/or immune cell recruitment and activation.

The terms “condition,” “disease,” and “disorder” are used interchangeably.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren’s syndrome, giant cell arteritis, progressive systemic sclerosis (scleroderma), ankylosing spondylitis, polymyositis, dermatomyositis, pemphigus, pemphigoid, diabetes (e.g., Type 1), myasthenia gravis, Hashimoto’s thyroiditis, Graves’ disease, Goodpasture’s disease, mixed connective tissue disease, sclerosing cholangitis, inflammatory bowel disease, Crohn’s disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener’s granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation. Inflammatory disease also includes postprandial inflammation, which is inflammation following the prolonged elevation of triglycerides occurring subsequent to ingestion of high-fat meals.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture’s disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture’s syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener’s granulomatosis, microscopic polyangiitis), uveitis, Sjogren’s syndrome, Crohn’s disease, Reiter’s syndrome, ankylosing spondylitis, Lyme disease, Guillain-Barré syndrome, Hashimoto’s thyroiditis, and cardiomyopathy.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enterocndocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, dyslipidemia, hyperlipidemia, metabolic syndrome, lipid-related metabolic disorder, hyperinsulinemia, insulin resistance, and obesity.

The term “pharmaceutically acceptable salt” as used herein refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, loweralkyl sulfonate and aryl sulfonate.

The term “agent” or “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

As used herein, the term “subject” refers to a human or non-human mammal or animal. Non-human mammals include livestock animals, companion animals, laboratory animals, and non-human primates. Non-human subjects also specifically include, without limitation, horses, cows, pigs, goats, dogs, cats, mice, rats, guinea pigs, gerbils, hamsters, mink, and rabbits. In some embodiments of the invention, a subject is referred to as a “patient.” In some embodiments, a patient or subject may be under the care of a physician or other health care worker, including, but not limited to, someone who has consulted with, received advice from or received a prescription or other recommendation from a physician or other health care worker.

The terms “decrease”, “reduced”, “reduction”, “inhibit” or “disrupt” are all used herein to mean a decrease by a statistically significant amount. In some embodiments, “reduce,” “reduction”, “decrease”, “inhibit” or “disrupt” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “alkyl” refers to a radical of a straight-chain or branched saturated hydrocarbon group having from 1 to 20 carbon atoms (“C₁₋₂₀ alkyl”) In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₀ alkyl (such as substituted C₁₋₆ alkyl, e.g., —CH₂F, —CHF₂, —CF₃ or benzyl (Bn)). An alkyl group may be branched or unbranched.

“Aralkyl” is a subset of “alkyl” and refers to an alkyl group substituted by an aryl group, wherein the point of attachment is on the alkyl moiety

As used herein, the term “alkoxy” refers to an alkyl group having an oxygen atom that connects the alkyl group to the point of attachment: i.e., alkyl—O—. As for the alkyl portions, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups are unsubstituted, but can be described, in some embodiments as substituted. “Substituted alkoxy” groups can be substituted with one or more moieties selected from halo, hydroxy, amino, alkylamino, nitro, cyano, and alkoxy.

The term “cycloalkyl” refers to cyclic alkyl radical having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The term “heteroalkyl,” as used herein, refers to an alkyl group, as defined herein, in which one or more of the constituent carbon atoms have been replaced by a heteroatom or optionally substituted heteroatom, e.g., nitrogen (e.g.,

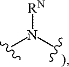

oxygen

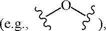

or sulfur

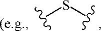

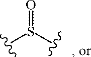

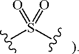

Heteroalkyl groups may be optionally substituted with one, two, three, or, in the case of alkyl groups of two carbons or more, four, five, or six substituents independently selected from any of the substituents described herein. Heteroalkyl group substituents include: (1) carbonyl; (2) halo; (3) C₆-C₁₀ aryl; and (4) C₃-C₁₀ carbocyclyl. A heteroalkylene is a divalent heteroalkyl group.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents (e.g., —F, —OH or -O(C₁₋₆ alkyl) . In certain embodiments, the aryl group is an unsubstituted C₆₋₁₄ aryl. In certain embodiments, the aryl group is a substituted C₆₋₁₄ aryl.

The term “aryloxy” refers to an —O—aryl substituent.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (-CHO), esters (e.g., —CO₂Rªª,—C(═O)SRªª, —C(═S)SR^{aa}), amides (e.g., —C(═O)N(R^{bb})₂, —C(═O)NR^{bb}SO₂Rªª, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “lipid” refers to a group of organic compounds that include, but are not limited to, esters of fatty acids and are characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids. The selection of the individual lipid components of the lipid.

The term “amino,” as used herein, represents —N(R^{N})₂, wherein each R^{N} is, independently, H, OH, NO₂, N(R^{N0})₂, SO₂OR^{N0}, SO₂R^{N0}, SOR^{N0}, an N-protecting group, alkyl, alkoxy, aryl, cycloalkyl, acyl (e.g., acetyl, trifluoroacetyl, or others described herein), wherein each of these recited R^{N} groups can be optionally substituted; or two R^{N} combine to form an alkylene or heteroalkylene, and wherein each R^{N0} is, independently, H, alkyl, or aryl. The amino groups of the disclosure can be an unsubstituted amino (i.e., —NH₂) or a substituted amino (i.e., —N(R^{N})₂).

The term “substituted” as used herein means at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, in some embodiments “substituted” means one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means one or more hydrogen atoms are replaced by a bond to an aminyl, cyano, hydroxyl, imino, nitro, oxo, thioxo, halo, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

The term “prodrug” refers to a compound having one or more cleavable groups that are cleaved by solvolysis, or under physiological conditions, to afford a parent compound (for example, a polyunsaturated fatty acid), said parent compound being pharmaceutically active in vivo. Such examples include, but are not limited to, esters such as alkyl or heteroalkyl ester derivatives, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₄ alkyl, C₁-C₆ alkyl, C₁—Cs alkyl, C₂-C₈ alkenyl, C₂-C₈ alkynyl, aryl, C₇-C₁₂ substituted aryl, and C₇-C₁₂ arylalkyl esters of the compounds described herein may be preferred.

The term “small molecule” refers to molecules, whether naturally-occurring or artificially created (e.g., via chemical synthesis) that have a relatively low molecular weight. Typically, a small molecule is an organic compound (e.g.., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

1. A method of treating a disorder in a subject resulting from activation of one or more xenobiotic sensor receptors in the subject by a xenobiotic agent, comprising administering to the subject an effective amount of a composition comprising a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion.

2. The method of claim 1, wherein the disorder is a disorder related to loss of intestinal homeostasis, an inflammatory disorder, or an autoimmune disease.

3. The method of claim 2, wherein the disorder related to loss of intestinal homeostasis is dyslipidemia, hyperlipidemia, metabolic syndrome, or a lipid-related metabolic disorder.

4. The method of claim 2, wherein the inflammatory disorder is post-prandial related inflammation.

5. The method of any one of the preceding claims, wherein administration of the composition to the subject results in inhibition of absorption, assembly, and/or transport of lipids, cholesterol, and/or microbial metabolites in the GI tract of the subject.

6. The method of claim 5, wherein the lipids are selected from diglycerides, triglycerides, fatty acids, phospholipids, cholesterol, cholesterol esters, glycolipids, bile acids, and microbial metabolites.

7. The method of claim 6, wherein the microbial metabolites are selected from glycosaccharides, glycolipids, free fatty acids, and microbial peptides.

8. The method of any one of the preceding claims, wherein the one or more xenobiotic sensor receptors are selected from pregnane X receptor (PXR) and constitutive active/androstane receptor (CAR).

9. The method of any one of the preceding claims, wherein the xenobotic agent has an EC50 of less than 10 µM in a cellular PXR assay.

10. The method of any one of the preceding claims, wherein the xenobiotic agent causes a gut-specific increase of MTP.

11. The method of any one of the preceding claims, wherein the xenobiotic agent activates or induces one or more cytochrome P450 enzymes.

12. The method of any one of the preceding claims, wherein the xenobiotic agent is a compound that is known to cause dyslipidemia, selected from amiodarone, β-blockers, loop diuretics, thiazide diuretics.

13. The method of any one of the preceding claims, wherein the xenobiotic agent is selected from phenothiazines, thioxanthenes, benztropines, corticosteroids, azoles, dihydropyridines, thiazolidinediones, thiazides, leptin, and leptin-mimetics.

14. The method of any one of the preceding claims, wherein the xenobiotic agent is selected from rifampicin, dexamethasone, ambrisentan, amlodipine, atorvastatin, bosentan, bumecainum, ciglitazone, clofenvinfosum, colforsin, demecolcine, dibunate, diclazuril, docusate, dronabinol, eburnamonine, ecopipamum, famprofazone, felodipine, flurometholone, fluvastatin, loratadine, lovastatin, metolazone, nilvadipine, nisoldipine, oxatomide, plicamycin, propiconazole, rifaximin, rimexolone, riodipine, simvastatin, spiroxatrine, teniliodona, terconanzole, testosterone, troglitazone, and zafirlukast.

15. The method of any one of the preceding claims, wherein the xenobiotic agent is an intestinal activator of STAT3 and/or MAPK, a non-nucleoside reverse transcriptase inhibitor, an antiretroviral agent, or a combination thereof.

16. The method of claim 15, wherein the xenobiotic agent is selected from lopinavir, atazanavir, fosamprcnavir, saquinavir, darunavir, tipranavir, efavirenz, nevirapine, tenofovir, abacavir, zidovudine, stavudine, ritonavir, amprenavir, indinavir, and nelfinavir.

17. The method of any one of claims 1-11, wherein the xenobiotic agent is an antipsychotic agent.

18. The method of claim 17, wherein the antipsychotic agent is selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, lcvomcpromazinc, lenperone, loxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine, zuclopenthixol, amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone, and zotepine.

19. The method of any one of the preceding claims, wherein the compound has the structure of Formula (I): 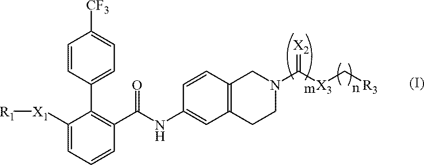
or a pharmaceutically acceptable salt, solvate, ester or hydrate thereof, wherein:
R₁ is alkyl, cycloalkyl, heterocyclyl, or R₄R₅NC(O)CH₂;
X₁ is a direct bond, O, S, N(R₆), C(O)NR₆, or N(R₆)C(O);
X₂ is O, N(R₆), or S;
X₃ is a direct bond, O, N(R₆) CH₂, arylene, or S;
R₃ is H, alkyl, alkoxy, heteroalkyl, cycloalkyl, heterocyclyl, aryl, heteroaryl, aryloxy, alkoxycarbonyl, arylcarbonyl, aryloxycarbonyl, —OH, —SH, or NR₄Rs;
R₄ and R₅ are, independently for each occurrence, H, alkyl, cycloalkyl, heterocyclyl, aryl, heteroaryl, heteroalkyl, aralkyl, aminocarbonyl, alkylcarbonyl, alkoxycarbonyl, arylcarbonyl, or aryloxycarbonyl;
R₆ is, independently for each occurrence, H or alkyl;
m is 0 or 1; and
n is an integer from 0 to 3;
provided that if m is 0, X₃ is a direct bond or CH₂.

20. The method of claim 19, wherein X₁ is O.

21. The method of any one of claims 19-20, wherein R₁ is alkyl.

22. The method of any one of claims 19-21, wherein R₁ is methyl.

23. The method of any one of claims 19-20, wherein R₁ and X₁ taken together form a moiety selected from (C₁-C₆-alkyl)—O—; 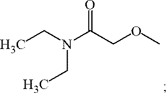 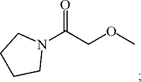 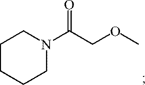 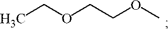 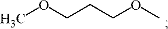 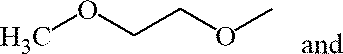 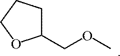
.

24. The method of claim 23, wherein R₁ and X₁ taken together form H₃C—O—, CH₃CH₂—O—, or (CH₃)₂CH—O—.

25. The method of any one of claims 19-24, wherein R₃ is aryl.

26. The method of claim 25, wherein R₃ is substituted or unsubstituted phenyl.

27. The method of any one of claims 19-25, wherein m is 1.

28. The method of any one of claims 19-27, wherein the moiety: 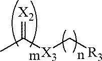
represents one of the following groups: 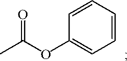 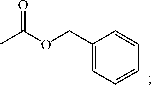 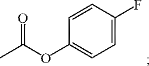 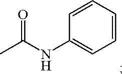 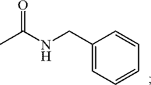 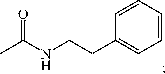 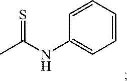 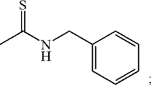 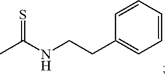 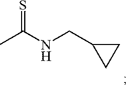 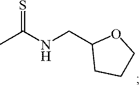 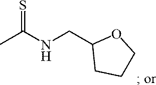 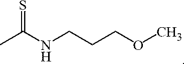
.

29. The method of any one of claims 1-18, wherein the compound has the structure of Formula (II): 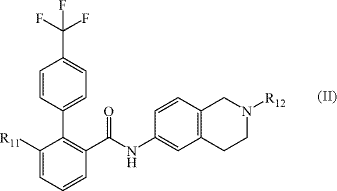
or a pharmaceutically acceptable salt, ester, isomer, or hydrate thereof, wherein:
R₁₁ is H or alkyl—O—, wherein the alkyl is substituted or unsubstituted; and
R₁₂ is substituted or unsubstituted heteroalkyl.

30. The method of claim 29, wherein R₁₁ is: 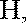 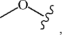 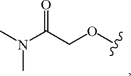 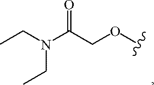 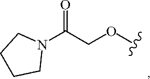 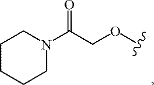 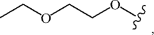 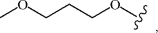 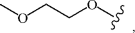 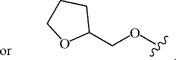
.

31. The method of any one of claims 29-30, wherein R₁₂ is: 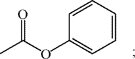 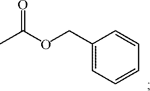 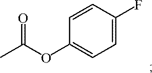 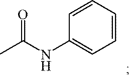 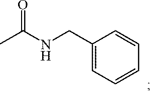 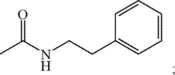 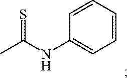 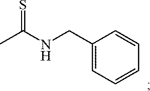 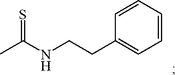 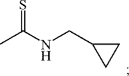 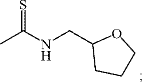 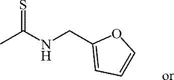 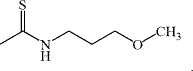
.

32. The method of any one of claims 1-31, wherein the compound has a structure selected from: 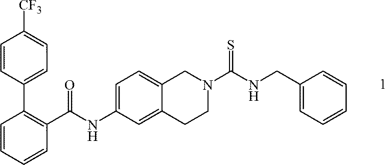 [SMILES: O=C(Nc1ccc2c(c1)CCN(C(=S)NCc1ccccc1)C2)c1ccccc1-c1ccc(C(F)(F)F)cc1] 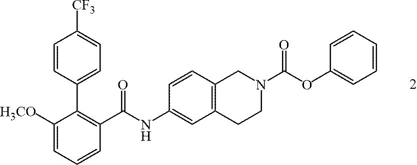 [SMILES: COc1cccc(C(=O)Nc2ccc3c(c2)CCN(C(=O)Oc2ccccc2)C3)c1-c1ccc(C(F)(F)F)cc1] 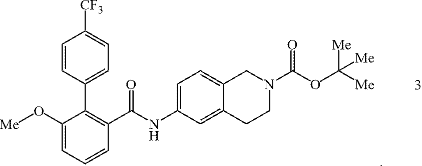 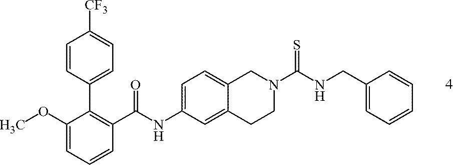 [SMILES: COc1cccc(C(=O)Nc2ccc3c(c2)CCN(C(=S)NCc2ccccc2)C3)c1-c1ccc(C(F)(F)F)cc1] 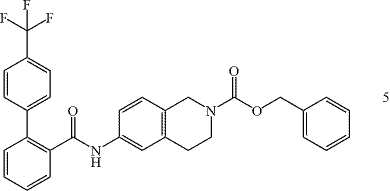 [SMILES: O=C(Nc1ccc2c(c1)CCN(C(=O)OCc1ccccc1)C2)c1ccccc1-c1ccc(C(F)(F)F)cc1] 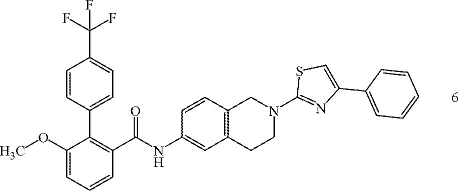 [SMILES: COc1cccc(C(=O)Nc2ccc3c(c2)CCN(c2nc(-c4ccccc4)cs2)C3)c1-c1ccc(C(F)(F)F)cc1] 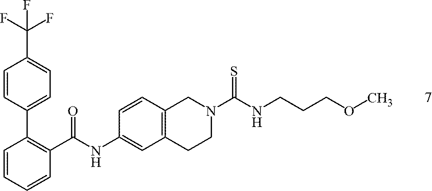 [SMILES: COCCCNC(=S)N1CCc2cc(NC(=O)c3ccccc3-c3ccc(C(F)(F)F)cc3)ccc2C1] 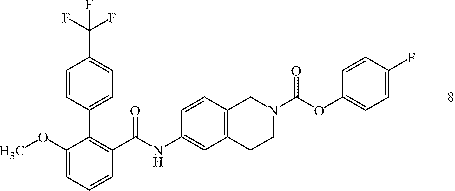 [SMILES: COc1cccc(C(=O)Nc2ccc3c(c2)CCN(C(=O)Oc2ccc(F)cc2)C3)c1-c1ccc(C(F)(F)F)cc1] 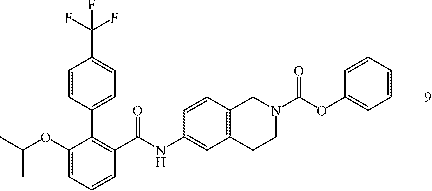 [SMILES: CC(C)Oc1cccc(C(=O)Nc2ccc3c(c2)CCN(C(=O)Oc2ccccc2)C3)c1-c1ccc(C(F)(F)F)cc1] 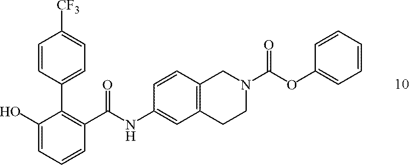 [SMILES: O=C(Nc1ccc2c(c1)CCN(C(=O)Oc1ccccc1)C2)c1cccc(O)c1-c1ccc(C(F)(F)F)cc1]
and pharmaceutically acceptable salts, solvates, hydrates, or esters thereof.

33. The method of any one of claims 1-18, wherein the compound is diethyl 2-((3-dimethylcarbamoyl-4-((4′-trifluoromethylbiphenyl-2-carbonyl)amino)phenyl)acetyloxymethyl)-2-phenylmalonate, or a pharmaceutically acceptable salt, solvate, hydrate, or ester thereof.

34. The method of any one of the preceding claims, wherein the compound is a GI selective MTP inhibitor.

35. The method of any one of the preceding claims, wherein the composition is formulated for immediate release.

36. The method of any one of the preceding claims, wherein the composition is formulated for extended release.

37. The method of any one of the preceding claims, wherein the compound is a systemically available inhibitor of MTP, NPC1N1, DGAT, MGAT, or ApoB secretion, and wherein the composition is formulated to limit oral bioavailability of the compound.

38. The method of any one of the preceding claims, wherein the compound is a systemically available inhibitor of MTP, NPC1N1, DGAT, MGAT, or ApoB secretion, and wherein the composition is formulated to promote GI selectivity of the compound.

39. The method of any one of the preceding claims, wherein the compound has less than 10% oral bioavailability.

40. The method of claim 39, wherein the compound has less than 3% oral bioavailability.

41. The method of claim 39, wherein the compound has less than 1% oral bioavailability.

42. The method of any one of the preceding claims, wherein the composition further comprises an additional therapeutic agent.

43. The method of claim 42, wherein the additional therapeutic agent modulates the absorption, assembly, and/or transport of lipids, cholesterol, and/or microbial metabolites in the GI tract of the subject.

44. The method of any one of claims 42-43, wherein the additional therapeutic agent is ezetimibe.

45. The method of any one of claims 42-44, wherein the additional therapeutic agent is a bile acid sequestrant.

46. The method of claim 45, wherein the bile acid sequestrant is selected from colestipol and cholestryramine.

47. The method of claim 42, wherein the additional therapeutic agent modulates production, secretion, transport, and/or homeostasis of lipids in the body of the subject.

48. The method of claim 47, wherein the additional therapeutic agent is an HMG-CoA reductase inhibitor, a fibric acid analog, nicotinic acid, an omega-3 fatty acid, or a PCSK9 inhibitor.

49. The method of any one of claims 42-43, wherein the additional therapeutic agent modulates the activation of the 5′ adenosine monophosphate-activated protein kinase (AMPK) pathway in the GI tract of the subject.

50. The method of claim 49, wherein the additional therapeutic agent is metformin, or a pharmaceutically acceptable salt or derivative thereof.

51. The method of any one of claims 42-43, wherein the additional therapeutic agent is a polyphenol.

52. The method of claim 51, wherein the polyphenol is epigallocatechin gallate, quercetin, apigenin, resveratrol, berberine, carnosol, curcumin, thienopyridone, salicylic acid, or a pharmaceutically acceptable salt, ester or derivative thereof.

53. The method of claim 49, wherein the additional therapeutic agent is a biguanide, a thiazolidinedione, or a Rho kinase inhibitor.

54. The method of claim 51, wherein the polyphenol is troglitazone, S17834 ([6,8-diallyl 5,7-dihydroxy 2-(2-allyl 3-hydroxy 4-methoxyphenyl)1-H benzo(b)pyran-4-one]), or MT 63-78.

55. The method of claim 49, wherein the additional therapeutic agent is an AMPK activator selected from: PF-249, PF-739, MK-8722, AICAR (N¹-(β-D-Ribofuranosyl)-5-aminoimidazole-4-carboxamide), A-769662 (6,7-Dihydro-4-hydroxy-3-(2′-hydroxy[1,1′-biphenyl]-4-yl)-6-oxo-thieno[2,3-b]pyridine-5-carbonitrile), cryptotanshinone (1,2,6,7,8,9-Hexahydro-1,6,6-trimethyl[1,2-b]furan-10,11-dione), RSV A 405 (2-[[4-(Diethylamino)-2-hydroxyphenyl]methylene]hydrazide-4-pyridinecarboxylic acid), ZLN 024 (2-[[2-(2-Bromo-4-methylphenoxy)ethyl]thio]pyrimidine), PT-1 (2-Chloro-5-[[5-[[5-(4,5-Dimethyl-2-nitrophenyl)-2-furanyl]methylene]-4,5-dihydro-4-oxo-2-thiazolyl]amino]benzoic acid), PF-06409577 (6-Chloro-5-[4-(1-hydroxycyclobutyl)phenyl]-1H-indole-3-carboxylic acid), α-lipoic acid, C-2 (benzimidazole, 5-(5-hydroxyl-isoxazol-3-yl)-furan-2-phosphonic acid), C-13 (prodrug of C-2, Compound 991, and ginsenosides.

56. The method of any one of claims 42-43, wherein the additional therapeutic agent is a modulator of CD1d.

57. The method of claim 56, wherein the CD1d modulator is an anti-inflammatory drug.

58. The method of claim 57, wherein the anti-inflammatory drug is a non-steroidal anti-inflammatory drug (NSAID), a corticosteroid (e.g., budesonide), an aminosalicylate (e.g., 5-ASA, and mesalamine), or an antibody (e.g., mepolizumab, adalimumab, golimumah, certolizumab, infliximab, tysbari, vedolizumab, and ustekinumab).

59. The method of claim 58, wherein the additional therapeutic agent is an immune system suppressing drug.

60. The method of claim 59, wherein the immune system suppressing drug is azathioprine, mercaptopurine, cyclosporine, or methotrexate.

61. The method of any one of claims 42-43, wherein the additional therapeutic agent is a TNF-α inhibitor, a dipeptydilpeptidase- 4 (DPP-4) inhibitor (e.g., sitagliptin), or a sodium-glucose co-transporter 2 (SGLT2) inhibitor.

62. The method of any one of the preceding claims, further comprising administering the xenobiotic agent to the subject.

63. The method of claim 62, wherein the composition and the xenobiotic agent are administered to the subject at the same time.

64. The method of claim 62, wherein the composition is administered to the subject after administration of the xenobiotic agent.

65. The method of claim 62, wherein the composition is administered to the subject before administration of the xenobiotic agent.

66. The method of any one of the preceding claims, wherein the effective amount of the composition contains a dose of the compound in the range of about 0.1 to about 5000 mg.

67. The method of claim 66, wherein the effective amount of the composition contains a dose of the compound in the range of about 1 to about 1200 mg body weight.

68. The method of claim 66, wherein the effective amount of the composition contains a dose of the compound in the range of about 5 to about 800 mg body weight.

69. The method of any one of the preceding claims, wherein the effective amount of the composition contains a dose of the compound in the range of about 0.1 to about 15 mg/kg body weight.

70. The method of any one of the preceding claims, wherein the effective amount of the composition contains a dose of the compound in the range of about 1 to about 5 mg/kg body weight.

71. The method of any one of the preceding claims, wherein the compound does not inhibit the activity of PXR.

72. The method of any one of the preceding claims, wherein the compound does not activate or induce a cytochrome P450 enzyme.

73. A pharmaceutical composition for use in the method of any one of claims 1-72.

74. The pharmaceutical composition of claim 73, wherein the compound that modulates the activity of microsomal triglyceride transfer protein (MTP), Neimann-Pick C 1-Like 1 protein (NPC1N1), diacylglycerol O-acyltransferase (DGAT), or monoacylglycerol acyltransferase (MGAT), or that blocks apolipoprotein B (ApoB) assembly and secretion is compound 2: 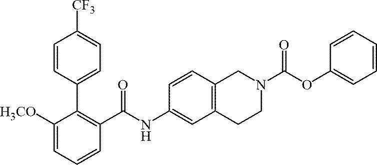 [SMILES: COc1cccc(C(=O)Nc2ccc3c(c2)CCN(C(=O)Oc2ccccc2)C3)c1-c1ccc(C(F)(F)F)cc1]
.

75. A kit comprising a compound that modulates the activity of microsomal triglyceride transfer protein (MTP), Neimann-Pick C1-Like 1 protein (NPC1N1), diacylglycerol O-acyltransferase (DGAT), or monoacylglycerol acyltransferase (MGAT), or that blocks apolipoprotein B (ApoB) assembly and secretion, as defined in any one of claims 1-41, and optionally an additional therapeutic agent as defined in any one of claims 42-61.

76. A pharmaceutical composition comprising a first compound that is an omega-3 fatty acid, or a prodrug thereof, and second compound that is an inhibitor of microsomal triglyceride transfer protein (MTP).

77. The pharmaceutical composition of claim 76, wherein the first compound is linoleic acid (ALA), eicosapentaenoic acid (EPA), docosapentaenoic acid (DPA), docosahexaenoic acid (DHA), or a prodrug thereof.

78. The pharmaceutical composition of any one of claims 76-77, wherein the first compound is two or more omega-3 fatty acids, or prodrugs thereof.

79. The pharmaceutical composition of any one of claims 76-78, wherein eicosapentaenoic acid, or a prodrug thereof, is present in an amount of about 70% to about 90%, by weight, of all fatty acids or prodrugs thereof present in the pharmaceutical composition.

80. The pharmaceutical composition of any one of claims 76-78, wherein docosapentaenoic acid, or a prodrug thereof, is present in an amount up to about 10%, by weight, of all fatty acids present in the pharmaceutical composition.

81. The pharmaceutical composition of claim 78, wherein docosapentaenoic acid, or a prodrug thereof, is present in an amount up to about 5%, by weight, of all fatty acids present in the pharmaceutical composition.

82. The pharmaceutical composition of claim 79, wherein docosapentaenoic acid, or a prodrug thereof, is present in an amount of about 5%, by weight, of all fatty acids present in the pharmaceutical composition.

83. The pharmaceutical composition of any one of claims 76-82, wherein the prodrug is an ester.

84. The pharmaceutical composition of claim 83, wherein the ester is a substituted or unsubstituted alkyl ester, or a substituted or unsubstituted heteroalkyl ester.

85. The pharmaceutical composition of claim 84, wherein the ester is an unsubstituted alkyl ester.

86. The pharmaceutical composition of claim 85, wherein the unsubstituted alkyl ester is a methyl ester, ethyl ester, propyl ester, isopropyl ester, n-butyl ester, or isobutyl ester.

87. The pharmaceutical composition of any one of claims 76-86, wherein the second compound is a small molecule, a polypeptide, or a polynucleotide.

88. The pharmaceutical composition of claim 87, wherein the second compound is a small molecule.

89. The pharmaceutical composition of any one of claims 76-88, wherein the second has the structure of Formula (I): 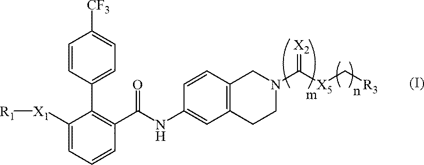
or a pharmaceutically acceptable salt, solvate, ester or hydrate thereof, wherein:
R₁ is alkyl, cycloalkyl, heterocyclyl, or R₄R₅NC(O)CH₂;
X₁ is a direct bond, O, S, N(R₆), C(O)NR₆, or N(R₆)C(O);
X₂ is O, N(R₆), or S;
X₃ is a direct bond, O, N(R₆) CH₂, arylene, or S;
R₃ is H, alkyl, alkoxy, heteroalkyl, cycloalkyl, heterocyclyl, aryl, heteroaryl, aryloxy, alkoxycarbonyl, arylcarbonyl, aryloxycarbonyl, —OH, —SH, or NR₄Rs;
R₄ and R₅ are, independently for each occurrence, H, alkyl, cycloalkyl, heterocyclyl, aryl, heteroaryl, heteroalkyl, aralkyl, aminocarbonyl, alkylcarbonyl, alkoxycarbonyl, arylcarbonyl, or aryloxycarbonyl;
R₆ is, independently for each occurrence, H or alkyl;
m is 0 or 1; and
n is an integer from 0 to 3;
provided that if m is 0, X₃ is a direct bond or CH₂.

90. The pharmaceutical composition of any one of claims 76-88, wherein the second compound has the structure of Formula (II): 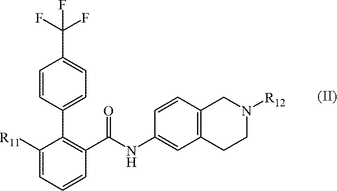
or a pharmaceutically acceptable salt, ester, isomer, or hydrate thereof, wherein:
R₁₁ is H or alkyl—O—, wherein the alkyl is substituted or unsubstituted; and
R₁₂ is substituted or unsubstituted heteroalkyl.

91. The pharmaceutical composition of any one of claims 76-90, wherein the second compound has a structure selected from: 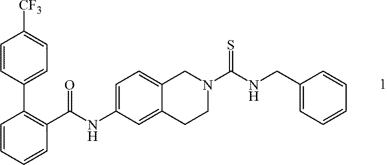 [SMILES: O=C(Nc1ccc2c(c1)CCN(C(=S)NCc1ccccc1)C2)c1ccccc1-c1ccc(C(F)(F)F)cc1] 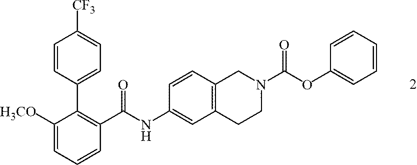 [SMILES: COc1cccc(C(=O)Nc2ccc3c(c2)CCN(C(=O)Oc2ccccc2)C3)c1-c1ccc(C(F)(F)F)cc1] 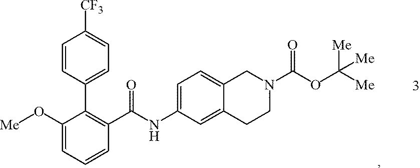 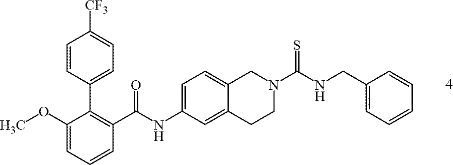 [SMILES: COc1cccc(C(=O)Nc2ccc3c(c2)CCN(C(=S)NCc2ccccc2)C3)c1-c1ccc(C(F)(F)F)cc1] 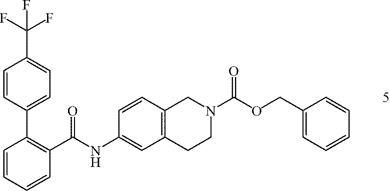 [SMILES: O=C(Nc1ccc2c(c1)CCN(C(=O)OCc1ccccc1)C2)c1ccccc1-c1ccc(C(F)(F)F)cc1] 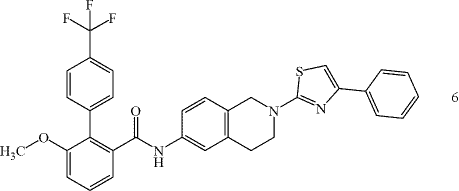 [SMILES: COc1cccc(C(=O)Nc2ccc3c(c2)CCN(c2nc(-c4ccccc4)cs2)C3)c1-c1ccc(C(F)(F)F)cc1] 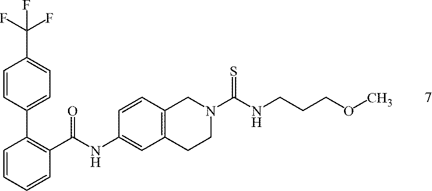 [SMILES: COCCCNC(=S)N1CCc2cc(NC(=O)c3ccccc3-c3ccc(C(F)(F)F)cc3)ccc2C1] 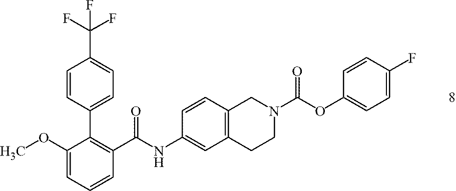 [SMILES: COc1cccc(C(=O)Nc2ccc3c(c2)CCN(C(=O)Oc2ccc(F)cc2)C3)c1-c1ccc(C(F)(F)F)cc1] 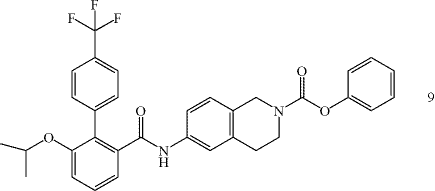 [SMILES: CC(C)Oc1cccc(C(=O)Nc2ccc3c(c2)CCN(C(=O)Oc2ccccc2)C3)c1-c1ccc(C(F)(F)F)cc1] 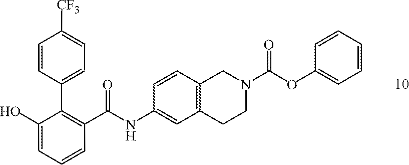 [SMILES: O=C(Nc1ccc2c(c1)CCN(C(=O)Oc1ccccc1)C2)c1cccc(O)c1-c1ccc(C(F)(F)F)cc1]
and pharmaceutically acceptable salts, solvates, hydrates, or esters thereof.

92. The pharmaceutical composition of any one of claims 76-91, wherein the second compound is diethyl 2-((3-dimethylcarbamoyl-4-((4′-trifluoromethylbiphenyl-2-carbonyl)amino)phenyl)acetyloxymethyl)-2-phenylmalonate, or a pharmaceutically acceptable salt, solvate, hydrate, or ester thereof.

93. The pharmaceutical composition of any one of claims 76-92, wherein the second compound is GI selective.

94. The pharmaceutical composition of any one of claims 76-93, wherein the composition is formulated for immediate release.

95. The pharmaceutical composition of any one of claims 76-93, wherein the composition is formulated for extended release.

96. The pharmaceutical composition of any one of claims 76-95, wherein the weight percent of the first compound in the composition is 1-5%, 5-7%, 7-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-93%, 93-95%, or 95-99%.

97. The pharmaceutical composition of any one of claims 76-96, wherein the weight percent of the second compound in the composition is 1-5%, 5-7%, 7-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-93%, 93-95%, or 95-99%.

98. The pharmaceutical composition of any one of claims 76-97, wherein the pharmaceutical composition comprises the first compound in an amount of about 10 mg to about 5000 mg.

99. The pharmaceutical composition of any one of claims 76-98, wherein the pharmaceutical composition comprises eicosapentaenoic acid in an amount of about 750 mg to about 950 mg.

100. The pharmaceutical composition of any one of claims 76-99, wherein the pharmaceutical composition comprises the second compound in an amount of about 1 mg to about 1200 mg.

101. A method of treating a disease or disorder in a subject in need thereof, comprising administering to the subject a first compound that is an omega-3 fatty acid, or a prodrug thereof, and second compound that is an inhibitor of microsomal triglyceride transfer protein (MTP).

102. The method of claim 101, wherein the disease or disorder is a metabolic disease.

103. The method of claim 102, wherein the metabolic disease is hypertriglyceridemia, mixed dyslipidemia, atherosclerosis, obesity, or diabetes.

104. The method of any one of claims 101-103, wherein the first compound is linoleic acid (ALA), eicosapentaenoic acid (EPA), docosahexaenoic acid (DHA), or a prodrug thereof.

105. The method of any one of claims 101-104, wherein the first compound is two or more omega-3 fatty acids, or prodrugs thereof.

106. The method of any one of claims 101-105, wherein the prodrug is an ester.

107. The method of claim 106, wherein the ester is a substituted or unsubstituted alkyl ester, or a substituted or unsubstituted heteroalkyl ester.

108. The method of claim 107, wherein the ester is an unsubstituted alkyl ester.

109. The method of claim 108, wherein the unsubstituted alkyl ester is a methyl ester, ethyl ester, propyl ester, isopropyl ester, n-butyl ester, or isobutyl ester.

110. The method of any one of claims 101-109, wherein the second compound is a small molecule, a polypeptide, or a polynucleotide.

111. The method of claim 110, wherein the second compound is a small molecule.

112. The method of any one of claims 101-111, wherein the second compound has the structure of Formula (I): 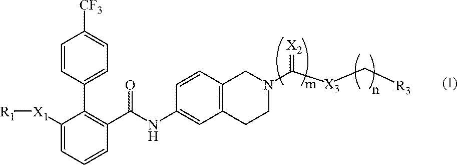
or a pharmaceutically acceptable salt, solvate, ester or hydrate thereof, wherein:
R₁ is alkyl, cycloalkyl, heterocyclyl, or R₄R₅NC(O)CH₂;
X₁ is a direct bond, O, S, N(R₆), C(O)NR₆, or N(R₆)C(O);
X₂ is O, N(R₆), or S;
X₃ is a direct bond, O, N(R₆) CH₂, arylene, or S;
R₃ is H, alkyl, alkoxy, heteroalkyl, cycloalkyl, heterocyclyl, aryl, heteroaryl, aryloxy, alkoxycarbonyl, arylcarbonyl, aryloxycarbonyl, —OH, —SH, or NR₄Rs;
R₄ and R₅ are, independently for each occurrence, H, alkyl, cycloalkyl, heterocyclyl, aryl, heteroaryl, heteroalkyl, aralkyl, aminocarbonyl, alkylcarbonyl, alkoxycarbonyl, arylcarbonyl, or aryloxycarbonyl;
R₆ is, independently for each occurrence, H or alkyl;
m is 0 or 1; and
n is an integer from 0 to 3;
provided that if m is 0, X₃ is a direct bond or CH₂.

113. The method of any one of claims 101-111, wherein the second compound has the structure of Formula (II): 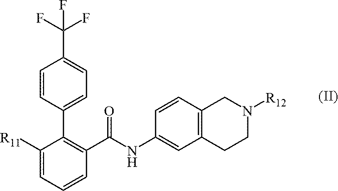
or a pharmaceutically acceptable salt, ester, isomer, or hydrate thereof, wherein:
R₁₁ is H or alkyl—O—, wherein the alkyl is substituted or unsubstituted; and
R₁₂ is substituted or unsubstituted heteroalkyl.

114. The method of any one of claims 101-113, wherein the second compound has a structure selected from: 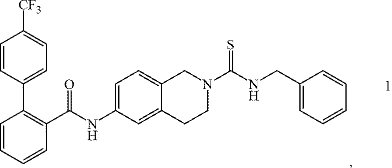 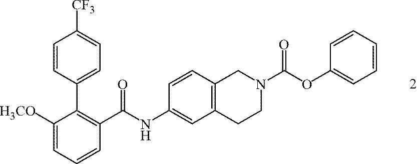 [SMILES: COc1cccc(C(=O)Nc2ccc3c(c2)CCN(C(=O)Oc2ccccc2)C3)c1-c1ccc(C(F)(F)F)cc1] 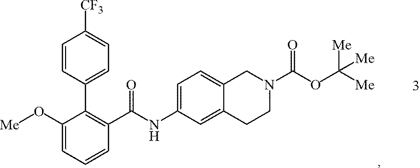 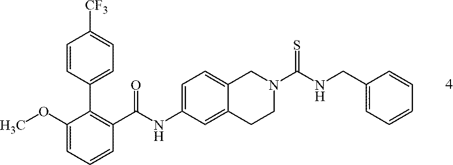 [SMILES: COc1cccc(C(=O)Nc2ccc3c(c2)CCN(C(=S)NCc2ccccc2)C3)c1-c1ccc(C(F)(F)F)cc1] 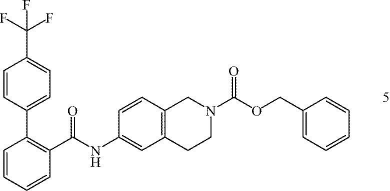 [SMILES: O=C(Nc1ccc2c(c1)CCN(C(=O)OCc1ccccc1)C2)c1ccccc1-c1ccc(C(F)(F)F)cc1] 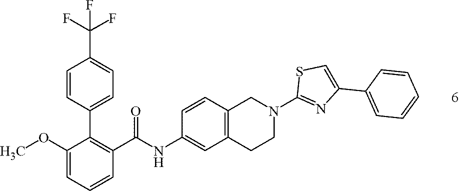 [SMILES: COc1cccc(C(=O)Nc2ccc3c(c2)CCN(c2nc(-c4ccccc4)cs2)C3)c1-c1ccc(C(F)(F)F)cc1] 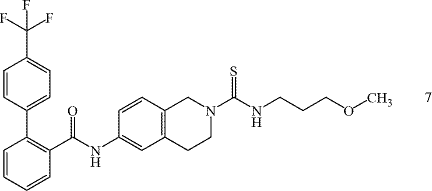 [SMILES: COCCCNC(=S)N1CCc2cc(NC(=O)c3ccccc3-c3ccc(C(F)(F)F)cc3)ccc2C1] 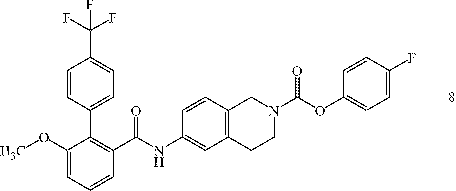 [SMILES: COc1cccc(C(=O)Nc2ccc3c(c2)CCN(C(=O)Oc2ccc(F)cc2)C3)c1-c1ccc(C(F)(F)F)cc1] 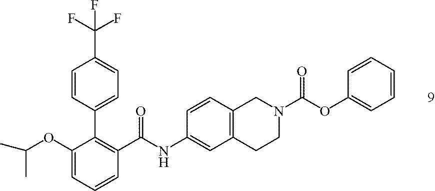 [SMILES: CC(C)Oc1cccc(C(=O)Nc2ccc3c(c2)CCN(C(=O)Oc2ccccc2)C3)c1-c1ccc(C(F)(F)F)cc1] 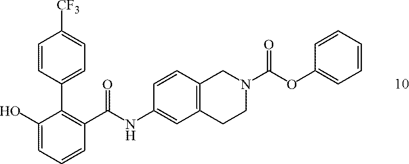 [SMILES: O=C(Nc1ccc2c(c1)CCN(C(=O)Oc1ccccc1)C2)c1cccc(O)c1-c1ccc(C(F)(F)F)cc1]
and pharmaceutically acceptable salts, solvates, hydrates, or esters thereof.

115. The method of any one of claims 101-114, wherein the second compound is diethyl 2-((3-dimethylcarbamoyl-4-((4′-trifluoromethylbiphenyl-2-carbonyl)amino)phenyl)acetyloxymethyl)-2-phenylmalonate, or a pharmaceutically acceptable salt, solvate, hydrate, or ester thereof.

116. The method of any one of claims 101-115, wherein the second compound is GI selective.

117. The method of any one of claims 101-116, wherein the first compound is administered prior to the second compound, e.g., less than hour prior, between 1-2 hours prior, between 2-4 hours prior, between 4-8 hours prior, between 8-16 hours prior, or between 16-48 hours prior.

118. The method of any one of claims 101-116, wherein the first compound is administered after the second compound, e.g., less than hour after, between 1-2 hours after, between 2-4 hours after, between 4-8 hours after, between 8-16 hours after, or between 16-48 hours after.

119. The method of any one of claims 101-116, wherein the first compound and the second compound are administered simultaneously.

120. The method of claim 101, comprising administering to the subject a pharmaceutical composition of any one of claims 76-100.

121. The method of any one of claims 101-120, wherein the subject has a history of acute heart failure, atrial fibrillation, hypoalbuminemia, or high inflammatory activity.

122. The method of any one of claims 101-121, wherein treating comprises reducing serum triglycerides in the subject.

123. The method of any one of claims 101-122, wherein the treatment comprises two or more administrations of the first compound and the second compound per day.

124. The method of any one of claims 101-122, wherein the treatment comprises one or more administrations of the first compound per day and two more administrations of the second compound per day.

125. The method of any one of claims 101-122, wherein the treatment comprises two or more administrations of the first compound per day and one more administrations of the second compound per day.

126. The method of any one of claims 101-125, wherein the efficacy of the first compound is improved by the second compound, as compared to the efficacy of the first compound alone.

127. The method of claim 126, wherein said efficacy of the first compound is the ability of the first compound to lower serum triglycerides in the subject.
